(19) United States
(12) Patent Application Publication
(10) Pub. No.: US 2009/0239872 A1
(43) Pub. Date: Sep. 24, 2009

(54) TREATMENT OF MESOTHELIOMA

(76) Inventor: John Arthur Hohneker, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/432,112

(22) Filed: Apr. 29, 2009

Related U.S. Application Data
(63) Continuation of application No. 10/534,572, filed on May 11, 2005, now abandoned.
(60) Provisional application No. 60/425,483, filed on Nov. 12, 2002.

(51) Int. Cl. A61K 31/502; C07D 401/04

(52) U.S. Cl. 514/248; 544/237

(57) ABSTRACT

The present invention relates to a method of treating a warm-blooded animal, especially a human, having mesothelioma, especially malignant mesothelioma, comprising administering to said animal a therapeutically effective amount of a 4-pyridylmethyl-phthalazine derivative.

This application is a continuation of U.S. Application No. 11/527,269 filed Sep. 26, 2006, which is a continuation of 10/534,572 filed May 11, 2005, which is a 371 of International Application No. PCT/EP03/12593, filed Nov. 11, 2003, which claims benefit of U.S. Provisional Application No. 60/425,483, filed Nov. 12, 2002.

The present invention relates to a method of treating a warm-blooded animal, especially a human, having mesothelioma, especially malignant mesothelioma, comprising administering to said animal a therapeutically effective amount of a 4-pyridylmethyl-phthalazine derivative, especially a compound of formula I as defined herein, alone or in combination with further therapeutic measures, for example, those defined herein; the use of a 4-pyridylmethyl-phthalazine derivative for the preparation of a medicament for the treatment of mesothelioma; and to a commercial package comprising a pharmaceutical composition together with instructions for its use in the treatment of mesothelioma.

Mesothelioma is a disease in which cancer (malignant) cells are found in the sac lining the chest (the pleura), the lining of the abdominal cavity (the peritoneum) or the lining around the heart (the pericardium). Most patients now being diagnosed with malignant mesothelioma have been exposed to asbestos in the 1940s, 50s, 60s, and 70s, because of the long latency period of asbestos diseases.

Early symptoms of the disease include shortness of breath, pain in the chest, or pain or swelling in the abdomen. For the full diagnosis of mesothelioma initially an x-ray or CT scan of the chest or abdomen is done. If further examination is warranted, thoracoscopy, peritoneoscopy or biopsy can be conducted. Pathology, the scientific study of cells, tissue, or fluid taken from the body, is an integral part of a mesothelioma diagnosis.

Surprisingly, it was found that 4-pyridylmethyl-phthalazine derivatives are useful for the treatment of mesothelioma.

4-Pyridylmethyl-phthalazine derivatives which a suitable for the present invention, their preparation and suitable pharmaceutical formulations containing the same are described in WO00/59509, EP02/04892, WO01/10859 and, especially, in U.S. Pat. No. 6,258,812, which are here incorporated by reference.

4-Pyridylmethyl-phthalazine derivatives and, in particular 4-pyridylmethyl-phthalazine derivatives of formula I,

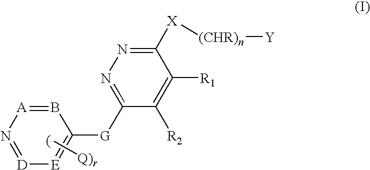

wherein the radicals and symbols have the meanings as defined below, the N-oxides of these 4pyridylmethyl-phthalazine derivatives, as well as the salts thereof, are tyrosine kinase inhibitors, which were designed to inhibit the vascular endothelial growth factor (VEGF) signal transduction by binding directly to the ATP-binding sites of VEGF receptors. Such 4-pyridylmethyl-phthalazine derivatives reduce the microvasculature and inhibit growth of primary tumors and metastases in animal models and are useful for treating diseases associated with deregulated angiogenesis, especially neoplastic diseases (solid tumors), such as breast cancer, cancer of the colon, lung cancer, especially small cell lung cancer, and cancer of the prostate.

Hence, the invention relates to a method of treating mesothelioma, especially malignant mesothelioma, comprising administering a therapeutically effective amount of a 4pyridyl-methyl-phthalazine derivative to a warm-blooded animal in need thereof, preferably of a therapeutically effective amount of a 4-pyridylmethyl-phthalazine derivative of formula I, wherein

r is 0 to 2,
n is 0 to 2,
m is 0 to 4,
R₁ and R₂ (i) are lower alkyl or
(ii) together form a bridge in subformula I*

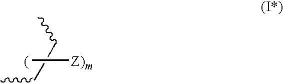

the binding being achieved via the two terminal carbon atoms, or
(iii) together form a bridge in subformula I**

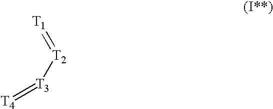

wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound,
or the salt of such compound having at least one salt-forming group.

The radicals and symbols as used in the definition of a compound of formula I have the meanings as disclosed in WO 98/35958 which publication is hereby incorporated into the present application by reference.

For example, 1-(4-chloroanilino)-4-(4-pyridylmethyl)phthalazine (also known as ZK222584), a compound of formula I, wherein r, n and m are each 0, R₁ and R₂ together form a bridge of subformula I*, A, B, D and E are each CH, G is methylene, X is imino, Y is 4chlorophenyl, and the bonds characterized by a wavy line are double bonds, is most specific for KDR, but can also inhibit Flt-1 and Flt-4 and has activity against other tyrosine kinase receptors, including c-Kit.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

A preferred compound of formula I is 1-(4-chloroanilino)-4-(4-pyridylmethyl)phthalazine. More preferably, 1-(4-chloroanilino)-4-(4pyridylmethyl)phthalazine is employed in the form of its succinate salt.

The term “mesothelioma” as used herein means in particular malignant mesothelioma, such as pleural mesothelioma, peritoneal mesothelioma, pericardial mesothelioma, epithelial mesothelioma, sarcomatous mesothelioma and biphasic mesothelioma.

The term “treatment” as used herein comprises the treatment of patients having mesothelioma or being in a pre-stage of said disease which effects the delay of progression of the disease in said patients.

For the treatment of mesothelioma a 4-pyridylmethyl-phthalazine derivative can be administered alone or in combination with other forms of treatments, e.g. surgery, in particular pleurectomy/decortication, pneumonectomy, extrapleural pneumonectomy, radiation therapy, in particular external radiation therapy or (internal radiation therapy, or administration of other therapeutic agents.

The person skilled in the pertinent art is fully enabled to select relevant test models to prove the hereinbefore and hereinafter mentioned beneficial effects on mesothelioma of a 4-pyridylmethyl-phthalazine derivative. The pharmacological activity of a 4-pyridylmethyl-phthalazine derivative may, for example, be demonstrated in a suitable clinical study. Suitable clinical studies are, for example, open label non-randomized, dose escalation studies in patients with advanced mesothelioma alone or in combination with additional therapeutic measures, e.g., those mentioned herein. The beneficial effects on mesothelioma can be determined directly through the results of such studies or by changes in the study design which are known as such to a person skilled in the art.

The effective dosage of a 4-pyridylmethyl-phthalazine derivative may vary depending on the particular compound or pharmaceutical composition employed, the mode of administration, the type of the mesothelioma being treated, the severity of the mesothelioma being treated and the co-medication. Thus, the dosage regimen of a 4-pyridylmethyl-phthalazine derivative is selected in accordance with a variety of factors including the route of administration and the renal and hepatic function of the patient. A physician, clinician or veterinarian of ordinary skill can readily determine and prescribe the effective amount of a 4-pyridylmethyl-phthalazine derivative required to prevent, counter or arrest the progress of the condition. Optimal precision in achieving concentration of the active ingredients within the range that yields efficacy without toxicity requires a regimen based on the kinetics of the active ingredients′ availability to target sites.

In the present invention, 1-(4-chloroanilino)-4-(4-pyridylmethyl)phthalazine, or a pharmaceutically acceptable salt thereof, can be administered twice or more daily, for example two or three times daily, on a continuous basis, alone, or during and subsequent to other therapies in reduced amounts. A daily oral administration of an amount in the range from 300 mg to 4000 mg, for example in the range from 300 mg/day to 2000 mg/day or 300 mg/day to 1000 mg/day, in particular 300, 500, 750, 1000, 1500 or 2000 mg/day, split into two doses, is contemplated as a pharmaceutically effective amount in the twice daily regimen. A 1000 mg/day dose is given as two 500 mg doses 6 to 12 hours apart, for example about 8 hours apart, and a 2000 mg/day dose is administered as two 1000 mg doses 6 to 8 hours apart, for example about 12 hours apart.

Alternatively, the present invention embraces a treatment regimen wherein 1-(4-chloroanilino)-4-(4pyridylmethyl)phthalazine is administered once daily at a dose in the range from 1000 mg/day to 1400 mg/day, particularly a dose of 1200 mg/day to 1300 mg/day, especially 1250 mg/day.

Moreover, the present invention provides a commercial package comprising a pharmaceutical composition together with instructions for its use in the treatment of mesothelioma.

The present invention also relates to the use of a 4-pyridylmethyl-phthalazine derivative for the preparation of a medicament for the treatment of mesothelioma.

What is claimed:

1. A method of treating mesothelioma comprising administering a therapeutically effective amount of a 4-pyridylmethyl-phthalazine derivative to a warm-blooded animal in need thereof.

2. Method according to claim 1 comprising administering a therapeutically effective amount of a 4-pyridylmethyl-phthalazine derivative of formula I 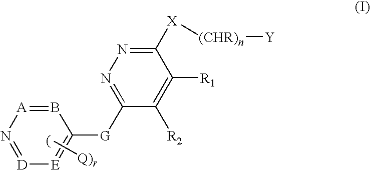
wherein
r is 0 to 2,
n is 0 to 2,
m is 0 to 4,
R₁ and R₂
(ii) together form a bridge in subformula I* 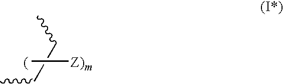
the binding being achieved via the two terminal carbon atoms, or
(iii) together form a bridge in subformula I** 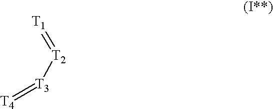
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not
more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or the salt of such compound having at least one salt-forming group, to a warm-blooded animal in need thereof.

3. Method of claim 2 wherein the 4-pyridylmethyl-phthalazine derivative of formula I is 1-(4-chloroanilino)-4-(4-pyridylmethyl)phthalazine.

4. Method according to any one of claim 1 wherein the disease is selected from pleural mesothelioma, peritoneal mesothelioma, pericardial mesothelioma, epithelial mesothelioma, sarcomatous mesothelioma and biphasic mesothelioma.

5. Method according to any one of claim 1 wherein the warm-blooded animal is a human.

6. Method according to claim 5 which comprises administering 1-(4-chloroanilino)-4-(4-pyridylmethyl)phthalazine, or a pharmaceutically acceptable salt thereof, to the patient on a once daily schedule at a dose in the range from 1000 mg/day to 1400 mg/day.

7. Method according to claim 6 wherein the once daily dose is 1200 mg/day to 1300 mg/day.

8. Method according to claim 6 wherein the once daily dose is 1250 mg/day.

9. A method of treating mesothelioma comprising administering a 4-pyridylmethyl-phthalazine derivative in an amount which is therapeutically effective against mesothelioma to a warm-blooded animal in need thereof in combination with surgery, in particular pleurectomy/decortication, pneumonectomy, extrapleural pneumonectomy, and/or radiation therapy, in particular external radiation therapy or internal radiation therapy,

10. A commercial package comprising a 4-pyridylmethyl-phthalazine derivative together with instructions for use thereof in the treatment of mesothelioma.

11. Method according to claim 2, wherein the 4-pyridylmethyl-phthalazine derivative is 1-(4-chloroanilino)-4-(4-pyridylmethyl)phthalazine or 1-(4-chloroanilino)-4-(4-pyridylmethyl)phthalazine in free form or in the form of a pharmaceutically acceptable salt.

12. Method according to claim 11, wherein the pharmaceutically acceptable salt is the succinate salt of the 4-pyridylmethyl-phthalazine derivative.
